(19) United States
(12) Patent Application Publication
(10) Pub. No.: US 2021/0137472 A1
(43) Pub. Date: May 13, 2021

(54) PROTECTION APPARATUS APPLIED TO RADIOTHERAPY TREATMENT COUCH AND RADIOTHERAPY TREATMENT COUCH

(71) Applicants: OUR UNITED CORPORATION, Xi'an City (CN); SMD Medical Co., Ltd., Wuhan City (CN)

(72) Inventors: Hui LIU, Xi'an City (CN); Jianguo HE, Xi'an City (CN); Fengjiao ZHANG, Xi'an City (CN); Shunwu ZHAO, Xi'an City (CN)

(21) Appl. No.: 17/089,371

(22) Filed: Nov. 4, 2020

(30) Foreign Application Priority Data
Nov. 13, 2019 (CN) 201921952287.2
Nov. 27, 2019 (CN) 201922082938.3

(51) Int. Cl. A61B 6/04; A61G 13/02

(52) U.S. Cl. CPC A61B 6/0421; A61G 13/02; A61G 2203/34; A61G 2210/00; A61G 2203/20

(57) ABSTRACT

A protection apparatus applied to a radiotherapy treatment couch and a radiotherapy treatment couch are provided. The protection apparatus includes: a protection panel and a status control mechanism connected to the protection panel, the protection panel being located in a to-be-protected area of the treatment couch and being perpendicular to a couch top of the treatment couch when in a use position, and the status control mechanism being configured to fix the protection panel to the use position or adjust the protection panel to a lay-down position. In the embodiments of the present disclosure, the status control mechanism may adjust the protection panel in the use position, or in the lay-down position, so that the protection panel may protect a patient when in the use position, and the protection panel may not hinder a movement of the patient when in the lay-down position.

### CROSS-REFERENCE TO RELATED APPLICATIONS

This application claims priority to the Chinese Patent Application No. 201921952287.2, filed on Nov. 13, 2019 and entitled “A protection apparatus applied to a radiotherapy treatment couch and a radiotherapy treatment couch”, and the Chinese Patent Application No. 201922082938.3, filed on Nov. 27, 2019 and entitled “An indication apparatus of a radiotherapy treatment couch, a treatment couch and a treatment equipment”, the disclosures of which are hereby incorporated by reference in their entirety.

### TECHNICAL FIELD

The present disclosure relates to the field of medical technology, specifically to a protection apparatus applied to a radiotherapy treatment couch.

### BACKGROUND

With the development of medical technology, radiotherapy has become an important means of tumor treatment. Generally, radiotherapy is performed by a patient lying on a radiotherapy treatment couch, and an operation of radiotherapy is realized by the movement of the radiotherapy treatment couch. Since the movement of the radiotherapy treatment couch may cause the patient to move, in addition to fixing the patient's position by a fixing apparatus, a protection apparatus is usually required to be set to prevent the patient from falling off the radiotherapy treatment couch.

Typically, the protection apparatus is installed on a side of the radiotherapy treatment couch, but the protection apparatus itself may prevent the patient from getting on and off the radiotherapy treatment couch. Therefore, requirements on an installation position and height of the protection apparatus are high, resulting in a complicated design of the protection apparatus of the radiotherapy treatment couch, and the use and operation of the protection apparatus of the radiotherapy treatment couch still hinders the patient.

### SUMMARY

In view of this, one of the technical problems solved by embodiments of the present disclosure is to provide a protection apparatus applied to a radiotherapy treatment couch and a radiotherapy treatment couch, to overcome at least part of the problems existing in the prior art.

An embodiment of the present disclosure provides a protection apparatus applied to a radiotherapy treatment couch, and the protection apparatus includes: a protection panel and a status control mechanism connected to the protection panel, the protection panel being located in a to-be-protected area of the treatment couch and being perpendicular to a couch top of the treatment couch when in a use position, and the status control mechanism being configured to fix the protection panel to the use position or adjust the protection panel to a lay-down position.

An embodiment of the present disclosure further provides a radiotherapy treatment couch, including the at least one protection apparatus.

As can be seen from the above technical solution, the protection apparatus of the embodiment of the present disclosure includes: a protection panel and a status control mechanism connected to the protection panel, the protection panel being located in a to-be-protected area of the treatment couch and being perpendicular to a couch top of the treatment couch when in a use position, and the status control mechanism be configured to fix the protection panel to the use position or adjust the protection panel to a lay-down position. In the embodiments of the present disclosure, the status control mechanism may adjust the protection panel in the use position, or in the lay-down position, so that the protection panel may protect a patient when in the use position, and the protection panel may not hinder a movement of the patient when in the lay-down position.

### BRIEF DESCRIPTION OF THE DRAWINGS

In order to more clearly explain the technical solution in embodiments of the present disclosure or the prior art, accompanying drawings required in the description of the embodiments or the prior art will be briefly introduced below. Obviously, the accompanying drawings in the following description are only some of the embodiments recorded in the embodiments of the present disclosure. For those skilled in the art, other accompanying drawings may also be obtained based on these accompanying drawings.

FIG. 1 is a schematic diagram of an embodiment of a protection apparatus applied to a radiotherapy treatment couch according to the present disclosure;

FIG. 2 is a schematic diagram of another embodiment of a protection apparatus applied to a radiotherapy treatment couch according to the present disclosure;

FIG. 3 is a schematic diagram of yet another embodiment of a protection apparatus applied to a radiotherapy treatment couch according to the present disclosure;

FIG. 4 is a schematic diagram of yet another embodiment of a protection apparatus applied to a radiotherapy treatment couch according to the present disclosure;

FIG. 5A and FIG. 5B are schematic diagrams of yet another embodiment of a protection apparatus applied to a radiotherapy treatment couch according to the present disclosure;

FIG. 6A is a schematic diagram of an embodiment of a radiotherapy treatment couch according to the present disclosure;

FIG. 6B is a schematic diagram of another embodiment of a radiotherapy treatment couch according to the present disclosure;

FIG. 7 is a schematic diagram of a sensing mechanism in yet another embodiment of a radiotherapy treatment couch according to the present disclosure;

FIG. 8 is a schematic diagram of a control circuit in yet another embodiment of a radiotherapy treatment couch according to the present disclosure; and

FIG. 9 is another schematic diagram of the control circuit in yet another embodiment of a radiotherapy treatment couch according to the present disclosure.

### DETAILED DESCRIPTION OF EMBODIMENTS

In order to enable those skilled in the art better understand the technical solution of the present application embodiment, in conjunction with the following drawings of the present application example embodiments, the technical solutions in the embodiments of the present application will be clearly and completely described, obviously, the described the present embodiments are merely part of application Example embodiment, rather than all embodiments. Based on the embodiments in the embodiments of the present application, all other embodiments obtained by those of ordinary skill in the art should fall within the protection scope of the embodiments of the present application.

Referring to FIG. 1, an embodiment of the present disclosure provides a protection apparatus 1 applied to a radiotherapy treatment couch. The protection apparatus 1 includes: a protection panel 11 and a status control mechanism 12 connected to the protection panel. The protection panel 11 is located in a to-be-protected area of the treatment couch and is perpendicular to a couch top 21 of the treatment couch when in a use position, and the status control mechanism is configure to fix the protection panel 12 to the use position or adjust the protection panel 11 to a lay-down position.

Specifically, the use position of the protection panel 11 is to be fixed perpendicular to the couch top 21 of the treatment couch, so as to protect a patient on the radiotherapy treatment couch. The lay-down position of the protection panel is to lay down the protection panel 11 so that the protection panel 11 may not hinder a movement of the patient. The lay-down position may be that the protection panel 11 is laid down in parallel to be parallel to a couch bottom 22 of the treatment couch, or the protection panel 11 is laid down vertically to be perpendicular to the couch bottom 22 of the treatment couch.

Specifically, the to-be-protected area of the treatment couch is a position on the periphery of the treatment couch and has a protective effect on preventing the patient from falling off the treatment couch. For example, two side edges of the treatment couch correspond to a position of the upper body of the patient.

In an embodiment of the present disclosure, the status control mechanism 12 may adjust the protection panel 11 to be in the use position or the lay-down position, so that the protection panel 11 may protect a patient when the protection panel 11 is in the use position, and the protection panel 11 may not hinder a movement of the patient when in the lay-down position.

In a specific embodiment of the present disclosure, a lower part of the protection panel 11 that is in contact with the couch top 21 of the treatment couch is adapted to a shape of the to-be-protected area of the treatment couch. Therefore, the protection panel 11 achieves a good contact relationship with the couch top 21 of the treatment couch with an adapted shape, which can better fence the couch top 21 of the treatment couch, and avoid patient rolling-off accidents.

In another specific embodiment of the present disclosure, referring to FIG. 2, the status control mechanism 12 includes: a status adjustment part 121 that moves in a direction perpendicular to the couch top of the treatment couch and a first fixing part 122, located in the to-be-protected area of the treatment couch, where when the status adjustment part 121 moves to a first position, the protection panel 11 is fixed by the first fixing part 122 to be perpendicular to the couch top of the treatment couch corresponding to the to-be-protected area of the treatment couch; and when the status adjustment part moves to a second position, the protection panel 11 is adjusted to the lay-down position.

In the embodiment of the present disclosure, the status of the protection panel 11 is adjusted to the use position or the lay-down position where the couch top 21 of the treatment couch corresponding to the to-be-protected area of the treatment couch is vertically fixed, through the movement of the status adjustment part 121 relative to the direction of the couch top of the treatment couch. In the embodiment of the present disclosure, when the status of the protection panel 11 by the first fixing part 122 is in the use position, the protection panel 11 is vertically fixed to the couch top of the treatment couch corresponding to the to-be-protected area of the treatment couch. Therefore, the structure of the status control mechanism 12 in the embodiment of the present disclosure enables the protection panel 11 to be conveniently adjusted to the use position or the lay-down position, which is simple to operate and easy to maintain.

In yet another specific embodiment of the present disclosure, in order to achieve a better vertical fixation of the protection panel 11 and the couch top of the treatment couch corresponding to the to-be-protected area of the treatment couch, the status control mechanism further includes: a second fixing part 123, located on the protection panel 11 at a position corresponding to the first position to assist in fixing the status adjustment part 121 to the first position.

In yet another specific embodiment of the present disclosure, the status adjustment part 121 includes: a lifting handle 1211 and at least one movement link 1212 connected to the lifting handle 1211, and the lifting handle 1211 is configured to receive an upward or downward force to drive the movement link 1212 to move between the first position and the second position.

Specifically, the lifting handle 1211 adopts a shape that is convenient for gripping, which facilitates the application of the upward or downward force by the lifting handle 1211.

In the embodiment of the present disclosure, when the status adjustment part 121 is in the first position, an upward force is applied by lifting the lifting handle 1211, thereby driving the at least one movement link 1212 connected to the lifting handle 1211 to move upward, so that the at least one movement link 1212 moves from the first position to the second position, and the status of the protection panel is switched from the use position to the lay-down position.

In the embodiment of the present disclosure, when the status adjustment part 121 is in the second position, a downward force is applied by pressing the lifting handle 1211, thereby driving the at least one movement link 1212 connected to the lifting handle to move downward, so that the at least one movement link 1212 moves from the second position to the first position, and the status of the protection panel is switched from the lay-down position to the use position.

Specifically, the first fixing part 122 is a fixing groove, the fixing groove is located on a side of the treatment couch where the to-be-protected area of the treatment couch is located, and the fixing groove corresponds to the movement link 1212, to receive the movement link 1212 such that the movement link moves through the fixing groove.

The movement link 1212 moves through the fixing groove, avoiding a left and right deviation when the movement link 1212 moves between the first position and the second position, which affects an operation control of status switching to the protection panel 11.

Referring to FIG. 3, the first fixing part 122 (fixing groove) is located on an outer side of the treatment couch, and the movement link 1212 passes through the fixing groove from the side of the protection panel 11. The installation method of the fixing groove in the embodiment of the present disclosure facilitates subsequent maintenance of the fixing groove.

Referring to FIG. 4, the first fixing part 122 (fixing groove) is located inside the side surface of the treatment couch, and the movement link 1212 passes through the fixing groove from the inside of the protection panel 11. The installation method of the fixing groove in the embodiment of the present disclosure is more beautiful.

Specifically, referring to FIG. 2, the second fixing part 123 is a fastening lock, the fastening lock is located on the protection panel 11 and corresponds to the movement link 1212, to assist in fixing the movement link 1212 to the first position.

In the embodiment of the present disclosure, the fastening lock used by the second fixing part 123 facilitates better fixing of the movement link 1212 to the first position, which is convenient for operation and subsequent maintenance.

Specifically, a number of the movement link 1212 is two, and the two movement links 1212 are respectively located at two ends of the lifting handle 1211. Therefore, it is convenient for the upward or downward force applied by the lifting handle 1211 to be well transmitted to the movement link 1212 and drive the movement link 1212 to move.

In yet another specific embodiment of the present disclosure, referring to FIG. 2, the status control mechanism 12 further includes: a third fixing part 124, configured to connect the protection panel 11 and the to-be-protected area of the treatment couch, when the third fixing part is in an upright status, the protection panel 11 is in the use position, or when the third fixing part is in a bent status, the protection panel 11 is in the lay-down position.

The third fixing part 124 may make the protection panel 11 realize status switching more stably.

For example, the third fixing part 124 is a hinge that is respectively fixed to the protection panel and the to-be-protected area of the treatment couch by two ends of a screw.

Referring to FIG. 5A and FIG. 5B, the status control mechanism 12 is located in a middle cavity of the protection panel 11, thereby making the appearance of the protection apparatus more beautiful.

A movement range of the status adjustment part 121 is between a lower edge of the protection panel 11 close to the couch top 21 of the treatment couch to and an upper edge of the protection panel 11 away from the couch top 21 of the treatment couch.

Referring to FIG. 1, an embodiment of the present disclosure also provides a radiotherapy treatment couch, which includes at least one protection apparatus 1 as described above.

The at least one protection apparatus 1 is disposed on at least one side surface of the couch top 21 of the radiotherapy treatment couch.

For example, there are two protection apparatuses 1 and they are respectively located on both sides of the couch top 21 of the treatment couch. As another example, there are three protection apparatuses 1, and they include located on both sides of the couch top 21 of the treatment couch respectively, and on a side surface of the patient's head or feet of the couch top 21 of the treatment couch.

In the embodiment of the present disclosure, the status control mechanism may adjust the protection panel to be in the use position or the lay-down position, so that the protection panel may protect a patient when the protection panel is in the use position, and the protection panel may not hinder a movement of the patient when in the lay-down position.

In a specific embodiment of the present disclosure, referring to FIG. 6A and FIG. 6B, the radiotherapy treatment couch further includes an indication apparatus 6, and the indication apparatus 6 includes: a sensing mechanism 61 and a display mechanism 62, the sensing mechanism 61 is connected to a protection panel 11 of the radiotherapy treatment couch and/or a status control mechanism 12 that controls the protection panel, and the sensing mechanism is configured to sense status information of the protection panel 11, and send the status information of the protection panel 11 to the display mechanism 62 for display.

Therefore, in the embodiment of the present disclosure, the status information of the protection panel may be displayed through the display mechanism, thereby preventing medical personnel from being able to understand the status of the protection panel 11 by observing the treatment couch.

In another specific embodiment of the present disclosure, the sensing mechanism 61 is further configured to send the status information of the protection panel 11 to a control system (not shown in the figure) of the radiotherapy equipment, and the control system (not shown in the figure) controls the radiotherapy equipment to perform radiotherapy based on the status information of the protection panel.

Specifically, the control system may control the movement of the radiotherapy couch and/or an activation of a radiation source apparatus (not shown in the figure) based on the status information.

For example, when the protection panel 11 is in the use position, the control system controls the radiotherapy couch to move, and/or when the protection panel 11 is in the use position, the control system controls an open source operation of the radiation source apparatus. When the protection panel 11 is in the lay-down position, the control system controls the radiotherapy couch to be unable to move, and/or, when the protection panel 11 is in the lay-down position, the control system controls the radiation source apparatus to be in a source off status.

The treating physician activates the radiotherapy equipment for radiotherapy, and if the protection panel is in the lay-down position, the display mechanism may display a warning for the status of the protection panel, where the warning may be provided by an indicator light of the treatment couch or the warning may be displayed on the display screen. In addition, when the protection panel is in the lay-down position, the control system controls the treatment couch not to move to avoid accidents. Or, when the protection panel is in the lay-down position, the control system controls the radiation source apparatus to turn off the source, and radiotherapy cannot be performed.

The display mechanism may be located on the radiotherapy equipment as shown in FIG. 6B, or may be a display located in a treatment control room. The display mechanism may also display a warning for the status of the protection panel, and prompt the treating physician to adjust the status of the protection panel so as to start radiotherapy. In the embodiment of the present disclosure, the control system of the radiotherapy equipment may be one or more. For example, the control system of the radiotherapy equipment includes a treatment couch control system and a radiation source apparatus control system, where the treatment couch control system is used to control an activation of the treatment couch, and the radiation source apparatus control system is used to control the activation of the radiation source apparatus. Then, the sensing mechanism may send the status information of the protection panel to the treatment couch control system and the radiation source apparatus control system, so as to realize the activation of the treatment couch and/or the radiation source apparatus. As another example, the radiotherapy equipment may include a control system that includes a plurality of sub-control systems, such as a treatment couch sub-control system and a radiation source apparatus sub-control system. The control system receives the status information of the protection panel and sends control instructions to the treatment couch sub-control system and the radiation source apparatus sub-control system respectively.

As another example, when the protection panel is in a working status or the lay-down position, the indication apparatus may also give a sound alarm to remind the treating physician to adjust the status of the protection panel.

Therefore, the embodiment of the present disclosure may control the status of the radiotherapy couch and/or the radiation source apparatus based on the status information of the protection panel 11, avoiding that since the protection panel 11 cannot protect the patient, and the operation of the radiotherapy couch and/or the radiation source apparatus causes the patient to slip off or suffer other injuries to cause the occurrence of medical malpractice.

In another specific embodiment of the present disclosure, referring to FIG. 7, the sensing mechanism 61 includes a sensor 611 and a control circuit 612 connected to the sensor, and the sensor 61 is connected to the protection panel 11 of the treatment couch and/or the status control mechanism 12 that controls the protection panel 11, and the sensor 61 is configured to sense a status of the protection panel 11 and transmit the sensed status to the control circuit 612 to convert the sensed status into the status information.

The present disclosure senses the status of the protection panel 11 through the sensor 611 and the control circuit 612 connected to the sensor 611, thereby realizing monitoring of a relative movement between the protection panel 11 of the treatment couch and the status control mechanism 12 through signal sensing and conversion, thereby realizing an acquisition of the status of the protection panel 11. The implementation of the present disclosure is simple in design and easy to maintain.

Specifically, the sensor is a non-contact sensor or a contact sensor.

The sensor is a non-contact sensor such as a displacement sensor. The non-contact sensor may sense the status of the protection panel 11 without contact, and its installation position is more flexible.

The sensor is a contact sensor, such as a proximity switch. Two contact ends of the contact sensor are respectively installed on any two contact ends of the radiotherapy couch, the protection panel 11, and the status control mechanism of the protection panel that can sense the status of the protection panel.

Since the contact sensor senses the status of the protection panel 11 through contact, its sensed accuracy is higher.

Referring to FIG. 2, the status control mechanism 12 includes: a status adjustment part 121 that moves in a direction perpendicular to the couch top of the treatment couch and a first fixing part 122 located in the to-be-protected area of the treatment couch, where when the status adjustment part 121 moves to a first position, the protection panel 11 is fixed by the first fixing part 122 to be perpendicular to the couch top of the treatment couch corresponding to the to-be-protected area of the treatment couch; and when the status adjustment part moves to a second position, the protection panel 11 is adjusted to the lay-down position.

In the embodiment of the present disclosure, the status of the protection panel 11 is adjusted to the use position or the lay-down position vertically fixed to the couch top 21 of the treatment couch corresponding to the to-be-protected area of the treatment couch through the movement of the status adjustment part 121 relative to the couch top of the treatment couch. In the embodiment of the present disclosure, when the status of the protection panel 11 by the first fixing part 122 is in the use position, the protection panel 11 is vertically fixed to the couch top of the treatment couch corresponding to the to-be-protected area of the treatment couch. Therefore, the structure of the status control mechanism 12 in the embodiment of the present disclosure enables the protection panel 11 to be conveniently adjusted to the use position or the lay-down position, which is simple to operate and easy to maintain.

In another specific embodiment of the present disclosure, in order to achieve a better vertical fixation of the protection panel 11 and the couch top of the treatment couch corresponding to the to-be-protected area of the treatment couch, the status control mechanism further includes: a second fixing part 123, located on the protection panel 11 at a position corresponding to the first position to assist in fixing the status adjustment part 121 to the first position.

In another specific embodiment of the present disclosure, the status adjustment part 121 includes: a lifting handle 1211 and at least one movement link 1212 connected to the lifting handle 1211, and the lifting handle 1211 is configured to receive an upward or downward force to drive the movement link 1212 to move between the first position and the second position.

Specifically, the lifting handle 1211 adopts a shape that is convenient for gripping, which facilitates the application of the upward or downward force by the lifting handle 1211.

In the embodiment of the present disclosure, when the status adjustment part 121 is in the first position, an upward force is applied by lifting the lifting handle 1211, thereby driving the at least one movement link 1212 connected to the lifting handle 1211 to move upward, so that the at least one movement link moves from the first position to the second position, and the status of the protection panel 11 is switched from the use position to the lay-down position.

In the embodiment of the present disclosure, when the status adjustment part 121 is in the second position, a downward force is applied by pressing the lifting handle 1211, thereby driving the at least one movement link 1212 connected to the lifting handle 1211 to move downward, so that the at least one movement link 1212 moves from the second position to the first position, and the status of the protection panel 11 is switched from the lay-down position to the use position.

Specifically, the first fixing part 122 is a fixing groove, the fixing groove is located on a side of the treatment couch where the to-be-protected area of the treatment couch is located, and the fixing groove corresponds to the movement link 1212, to receive the movement link 1212 such that the movement link 1212 moves through the fixing groove.

The movement link 1212 moves through the fixing groove, avoiding a left and right deviation when the movement link 1212 moves between the first position and the second position, which affects an operation control of status switching to the protection panel 11.

Referring to FIG. 3, the first fixing part 122 (fixing groove) is located on an outer side of the treatment couch, and the movement link 1212 passes through the fixing groove from the side of the protection panel 11. The installation method of the fixing groove in the embodiment of the present disclosure facilitates subsequent maintenance of the fixing groove.

Referring to FIG. 4, the first fixing part 122 (fixing groove) is located inside the side surface of the treatment couch, and the movement link 1212 passes through the fixing groove from the inside of the protection panel 11. The installation method of the fixing groove in the embodiment of the present disclosure has a great aesthetic value

Specifically, referring to FIG. 2, the second fixing part 123 is a fastening lock, the fastening lock is located on the protection panel 11 and corresponds to the movement link 1212, to assist in fixing the movement link 1212 to the first position.

In the embodiment of the present disclosure, the fastening lock used by the second fixing part 123 facilitates better fixing of the movement link 1212 to the first position, which is convenient for operation and subsequent maintenance.

Specifically, the number of the movement link 1212 is two, and the two movement links 1212 are respectively located at two ends of the lifting handle 1211. Therefore, it is convenient for the upward or downward force applied by the lifting handle 1211 to be well transmitted to the movement link 1212 and drive the movement link 1212 to move.

In yet another specific embodiment of the present disclosure, referring to FIG. 2, the status control mechanism 12 further includes: a third fixing part 124, configured to connect the protection panel 11 and the to-be-protected area of the treatment couch, when the third fixing part is in an upright status, the protection panel 11 is in the use position, or when the third fixing part is in a bent status, the protection panel 11 is in the lay-down position.

The third fixing part 124 may make the protection panel 11 realize status switching more stably.

For example, the third fixing part 124 is a hinge that is respectively fixed to the protection panel and the to-be-protected area of the treatment couch by two ends of a screw.

Referring to FIG. 5A and FIG. 5B, the status control mechanism 12 is located in a middle cavity of the protection panel 11, thereby making the appearance of the protection apparatus more beautiful.

A movement range of the status adjustment part 121 is between a lower edge of the protection panel 11 close to the couch top 21 of the treatment couch to and an upper edge of the protection panel 11 away from the couch top 21 of the treatment couch.

Specifically, the sensor 611 is a pressure sensor 6111, and the pressure sensor 6111 is set at the first position or the second position, the status information of the protection panel 11 is obtained by sensing a movement status of the status control mechanism 12. Because of its small size and convenient setting, the pressure sensor 6111 may sense the movement of the status control mechanism by sensing a pressure change caused by the movement of the movement link 12 at the first position or the second position, thereby obtaining the status information of the protection panel 11.

In yet another specific embodiment of the present disclosure, referring to FIG. 8, the control circuit 612 includes: an analog-digital conversion circuit 6121 that converts an analog pressure status value sent by the pressure sensor 6111 into a digital status value.

In the present disclosure, an analog signal sensed by the pressure sensor 6111 is converted into a digital signal by the analog-digital conversion circuit 6121, and transmitted to the display mechanism 62 for display, thereby improving the accuracy of signal transmission control.

In yet another specific embodiment of the present disclosure, referring to FIG. 9, the control circuit 612 further includes a noise filtering circuit 6122 configured to filter noise in an analog pressure status value sent by a pressure sensor 6111, and send the noise-filtered analog pressure status value to the analog-digital conversion circuit 6121.

In the present disclosure, the noise filtering circuit 6122 performs noise filtering on the analog pressure status value sent by the pressure sensor 6111, avoiding inaccurate signal detection caused by noise interference in the analog signal, and improving signal accuracy.

In yet another specific embodiment of the present disclosure, the pressure sensor 6111 includes a plurality of sub-pressure sensors, which are set on the same protection panel for mutual sensing and verification, or the plurality of sub-pressure sensors are respectively set at contact positions of different protection panels and the status control mechanism of the protection panels to sense the status information of the different protection panels.

Specifically, the display mechanism 62 includes: an LED indicator light and a display screen.

The present disclosure displays the status information of the protection panel 11 through the LED indicator and/or the display screen, which can provide medical personnel with an intuitive display effect and is easy to install and maintain.

The LED indicator light and/or the display screen receives the status information sent by the plurality of sub-pressure sensors, and performs different displays based on the status information.

For example, the LED indicator light may indicate the working status of the protection panel in different colors. For example, when the protection panel is in the working status, the LED indicator light may display green, and when the protection panel is in the lay-down position, the LED indicator light may display red. The LED indicator light may also indicate the working status of the protection panel in a display method. For example, when the protection panel is in the working status, the LED indicator light may be in stable display, and when the protection panel is in the lay-down position, the LED indicator light may be flashing display.

Therefore, the present disclosure may set a plurality of sub-pressure sensors for the same protection panel to verify mutual sensing. The present disclosure may also display differently for different protection panels, and display the status information of the different protection panels through the display mechanism, avoiding inconvenience in use caused by the need for medical personnel to comprehensively observe the radiotherapy treatment couch, and further improving use experience of the radiotherapy treatment couch. Finally, it should be noted that the above embodiments are only used to illustrate the technical solution of the embodiments of the present disclosure, rather than limiting it; although the present disclosure has been described in detail with reference to the foregoing embodiments, those of ordinary skill in the art should understand: it is still possible to modify the technical solution described in the foregoing embodiments, or equivalently replace some of the technical features therein; and these modifications or replacements do not make the essence of the corresponding technical solution deviate from the spirit and scope of the technical solution of the embodiments of the present disclosure.

What is claimed is:

1. A protection apparatus applied to a radiotherapy treatment couch, the protection apparatus comprising: a protection panel and a status control mechanism connected to the protection panel, the protection panel being located in a to-be-protected area of the treatment couch and being perpendicular to a couch top of the treatment couch when in a use position, and the status control mechanism being configured to fix the protection panel to the use position or adjust the protection panel to a lay-down position.

2. The protection apparatus according to claim 1, wherein a lower part of the protection panel that is in contact with the couch top of the treatment couch is adapted to a shape of the to-be-protected area of the treatment couch.

3. The protection apparatus according to claim 1, wherein the status control mechanism comprises: a status adjustment part that moves in a direction perpendicular to the couch top of the treatment couch, and a first fixing part located in the to-be-protected area of the treatment couch, wherein when the status adjustment part moves to a first position, the protection panel is fixed by the first fixing part to be perpendicular to the couch top of the treatment couch corresponding to the to-be-protected area of the treatment couch; and when the status adjustment part moves to a second position, the protection panel is adjusted to the lay-down position.

4. The protection apparatus according to claim 3, wherein the status control mechanism further comprises: a second fixing part, located on the protection panel at a position corresponding to the first position to assist in fixing the status adjustment part to the first position.

5. The protection apparatus according to claim 3, wherein the status adjustment part comprises: a lifting handle and at least one movement link connected to the lifting handle, the lifting handle is configured to receive an upward or downward force to drive the movement link to move between the first position and the second position.

6. The protection apparatus according to claim 5, wherein the first fixing part is a fixing groove, and the fixing groove is located on a side of the treatment couch where the to-be-protected area of the treatment couch is located, and the fixing groove corresponds to the movement link, to receive the movement link such that the movement link moves through the fixing groove.

7. The protection apparatus according to claim 6, wherein the second fixing part is a fastening lock, and the fastening lock is located on the protection panel and corresponds to the movement link, to assist in fixing the movement link to the first position.

8. The protection apparatus according to claim 7, wherein a number of the movement link is two, and the two movement links are respectively located at two ends of the lifting handle.

9. The protection apparatus according to claim 3, wherein the status control mechanism further comprises: a third fixing part, configured to connect the protection panel and the to-be-protected area of the treatment couch, wherein when the third fixing part is in an upright status, the protection panel is in the use position, or when the third fixing part is in a bent status, the protection panel is in the lay-down position.

10. The protection apparatus according to claim 3, wherein the status adjustment part further comprises a position sensing element by which whether the protection panel is adjusted to be in the use position or in the lay-down position by the status adjustment part is sensed.

11. The protection apparatus according to claim 5, wherein the status control mechanism further comprises: a first guide part provided on at least one side of the lifting handle and a second guide part provided on the protection panel, wherein the first guide part and the second guide part are opposite and fit with each other to limit a movement direction of the lifting handle.

12. A radiotherapy treatment couch, comprising at least one protection apparatus according to claim 1.

13. The radiotherapy treatment couch according to claim 12, wherein the at least one protection apparatus is disposed on at least one side surface of a couch top of the radiotherapy treatment couch.

14. The radiotherapy treatment couch according to claim 12, wherein the radiotherapy treatment couch further comprises an indication apparatus, and the indication apparatus comprises: a sensing mechanism and a display mechanism, the sensing mechanism is connected to a protection panel of the radiotherapy treatment couch and/or a status control mechanism that controls the protection panel, and the sensing mechanism is configured to sense status information of the protection panel, and send the status information of the protection panel to the display mechanism for display.

15. The radiotherapy treatment couch according to claim 14, wherein the sensing mechanism is further configured to send the status information of the protection panel to a control system of a radiotherapy equipment, and the control system controls the radiotherapy equipment to perform radiotherapy based on the status information of the protection panel.

16. The radiotherapy treatment couch according to claim 14, wherein the sensing mechanism comprises a sensor and a control circuit connected to the sensor, and the sensor is connected to the protection panel of the treatment couch and/or a status control mechanism that controls the protection panel, and the sensor is configured to sense a status of the protection panel and transmit the sensed status to the control circuit to convert the sensed status into the status information.

17. The radiotherapy treatment couch according to claim 16, wherein the status control mechanism comprises: a status adjustment part that moves in a direction perpendicular to a couch top of the treatment couch, and a first fixing part located in a to-be-protected area of the treatment couch, wherein when the status adjustment part moves to a first position, the protection panel is fixed by the first fixing part to be perpendicular to the couch top of the treatment couch corresponding to the to-be-protected area of the treatment couch; and when the status adjustment part moves to a second position, the protection panel is adjusted to a lay-down position; and wherein the sensor is a pressure sensor, and the pressure sensor is provided at the first position or the second position and configured to obtain the status information of the protection panel by sensing a movement status of the status control mechanism.

18. The radiotherapy treatment couch according to claim 16, wherein the control circuit comprises: an analog-digital conversion circuit that converts an analog pressure status value sent by a pressure sensor into a digital status value.

19. The radiotherapy treatment couch according to claim 16, wherein the control circuit further comprises: a noise filtering circuit configured to filter noise in an analog pressure status value sent by a pressure sensor, and send the noise-filtered analog pressure status value to the analog-digital conversion circuit.

20. The radiotherapy treatment couch according to claim 14, wherein the display mechanism comprises: at least one of an LED indicator light and a display screen.
